(19) United States
(12) Patent Application Publication
(10) Pub. No.: US 2026/0280355 A1
(43) Pub. Date: Sep. 17, 2026

(54) DEVICES AND METHODS FOR RELAYING POWER TO AN ELECTRONIC DEVICE

(71) Applicant: THE ALFRED E. MANN FOUNDATION FOR SCIENTIFIC RESEARCH, VALENCIA, CA (US)

(72) Inventors: Anthony Ng, Santa Clarita, CA (US); Leslie Farkas, Ventura, CA (US)

(21) Appl. No.: 19/549,621

(22) Filed: Feb. 25, 2026

Related U.S. Application Data
(60) Provisional application No. 63/770,267, filed on Mar. 11, 2025.

(51) Int. Cl. H02J 50/50; A61N 1/378; H02J 50/10; H02J 50/15

(52) U.S. Cl. CPC H02J 50/50; A61N 1/3787; H02J 50/10; H02J 50/15

(57) ABSTRACT

A wireless power relay for wirelessly relaying power to an electronic device includes a power receiver configured to wirelessly receive power; a power transmitter configured to wirelessly transmit power; and a power converter operatively coupled between the power receiver and the power transmitter, the power converter being configured to convert first power at a first frequency wirelessly received by the power receiver, and to apply the converted power to the power transmitter such that the power transmitter wirelessly transmits second power at a second frequency different from the first frequency.

### CROSS-REFERENCE TO RELATED APPLICATION

The present application claims priority to, and the benefit of, U.S. Provisional Patent Application No. 63/770,267, filed on Mar. 11, 2025, the entire content of which is incorporated herein by reference.

### BACKGROUND

### 1. Field

The present disclosure relates to systems and methods for wirelessly relaying power to an electronic device, and more particularly, to wirelessly relaying power from a power base station to an implantable medical device.

### 2. Description of the Related Art

Implantable medical devices (IMDs) can be subcutaneously implanted under a patient's skin and configured to provide one or more functions useful in diagnosing and/or treating a medical condition. For example, an implantable pulse generator (IPG) can be used to provide electrical stimulation to a patient's vagus nerve to treat epilepsy or depression in the patient. Some IMDs do not include a primary battery and instead rely on wirelessly receiving power from an external charger, which can be stored in a secondary battery of the IMD or used to operate the IMD. However, such external chargers are often large and heavy, which reduces their mobility and the convenience and comfort of the patients that use them. These external chargers can also require active involvement from the patient (e.g., positioning, holding, and/or interacting with the external charger) during operation. Charging times for IMDs can be long (e.g., greater than an hour), which can be a significant inconvenience for the patient.

There is a continuing need to improve systems and processes for providing power to IMDs. It would be desirable to efficiently and passively wirelessly transfer power to a patient's IMD without requiring active involvement from the patient and in a manner that improves the comfort, convenience, and mobility of the patient during the transfer of power. It is in view of this technical background that the present disclosure is provided.

This background section is provided only for purposes of introducing certain background material relating to the present disclosure and, thus, is not an admission of prior art.

### SUMMARY

According to an aspect, the technology relates to a wireless power relay for wirelessly relaying power to an electronic device, the wireless power relay including a power receiver configured to wirelessly receive power; a power transmitter configured to wirelessly transmit power; and a power converter operatively coupled between the power receiver and the power transmitter, the power converter being configured to convert first power at a first frequency wirelessly received by the power receiver, and to apply the converted power to the power transmitter such that the power transmitter wirelessly transmits second power at a second frequency different from the first frequency.

In some examples, the second frequency is lower than the first frequency.

In some examples, the second frequency is higher than the first frequency.

In some examples, the electronic device includes an implantable medical device.

In some examples, the wireless power relay includes a biocompatible adhesive configured to attach the wireless power relay to the skin of a patient.

In some examples, a wireless power relay system includes the wireless power relay, and a wearable garment configured to support and position the wireless power relay in proximity to the implantable medical device.

In some examples, the power converter is configured to convert the first power and to apply the converted power to the power transmitter without storing power in a battery.

In some examples, the wireless power relay does not include a battery.

In some examples, the wireless power relay includes a battery.

In some examples, the power receiver includes a receiver antenna, and the power transmitter includes a transmitter coil.

In some examples, the power converter is configured: to convert a first AC current, having the first frequency and being generated in the receiver antenna in response to the receiver antenna wirelessly receiving the first power, to a second AC current having the second frequency; and to provide the second AC current to the transmitter coil to cause the transmitter coil to wirelessly transmit the second power.

In some examples, the power receiver and the power transmitter are respectively positioned in first and second compartments that are at least partially separated by a power separator configured to at least partially separate the first power from the second power.

In some examples, at least one of the first power or the second power includes electromagnetic power or magnetic power, and the power separator includes an electrically conductive element configured to at least partially block electromagnetic power and magnetic power from transferring between the first and second compartments.

In some examples, the second power includes ultrasonic power, and the power separator includes an acoustic separator configured to at least partially block ultrasonic power from transferring from the second compartment into the first compartment.

In some examples, the wireless power relay includes a beacon transmitter configured to wirelessly transmit a beacon signal.

In some examples, a wireless power transfer system, includes the wireless power relay of claim 1; and a base station configured to wirelessly transmit the first power at the first frequency to the wireless power relay.

In some examples, the wireless power transfer system includes the electronic device, the electronic device being configured to wirelessly receive the second power from the wireless power relay.

In some examples, the base station is configured to control a direction of the first power wirelessly transmitted by the base station.

In some examples, the wireless power relay includes a beacon transmitter configured to wirelessly transmit a beacon signal, and the base station includes a phased array antenna configured to wirelessly receive the beacon signal, the base station being configured to wirelessly transmit the first power, via the phased array antenna, based on the beacon signal.

In some examples, a wireless power transfer system, includes the wireless power relay; and the electronic device, the electronic device including an implantable medical device configured to wirelessly receive the second power from the wireless power relay.

In some examples, the implantable medical device is an implantable pulse generator configured to provide stimulation to tissue.

In some examples, the power receiver includes an antenna configured to receive electromagnetic power.

In some examples, the power transmitter includes a transmitter coil configured to generate an alternating magnetic field.

In some examples, the power transmitter includes a transmitter antenna configured to generate electromagnetic power.

In some examples, the power transmitter includes an ultrasonic transducer configured to generate ultrasonic power.

According to an aspect, the technology relates to a wireless power relay for wirelessly relaying power to an electronic device, the wireless power relay including a receiver antenna configured to generate a first AC current in response to receiving first electromagnetic power; a transmitter coil configured to generate an alternating magnetic field in response to being driven with a second AC current; and a frequency converter operatively coupled between the receiver antenna and the transmitter coil, the frequency converter being configured to convert the first AC current, having a first frequency, to the second AC current having a second frequency different from the first frequency, and to drive the transmitter coil with the second AC current.

In some examples, the electronic device includes an implantable medical device.

In some examples, the second frequency is lower than the first frequency.

In some examples, the second frequency is higher than the first frequency.

In some examples, the wireless power relay does not include a battery.

In some examples, the wireless power relay includes a battery.

In some examples, the wireless power relay includes a biocompatible adhesive configured to attach the wireless power relay to the skin of a patient.

In some examples, a wireless power transfer system includes the wireless power relay; and a base station configured to transmit the first electromagnetic power to the wireless power relay.

In some examples, the wireless power transfer system includes an implantable medical device configured to wirelessly receive the alternating magnetic field and to convert the alternating magnetic field into an electric current.

In some examples, a wireless power transfer system includes the wireless power relay; and an implantable pulse generator configured to wirelessly receive the alternating magnetic field.

According to an aspect, the technology relates to a method for wirelessly relaying power to an electronic device, the method including wirelessly receiving, by a wireless power relay, power having a first frequency; and wirelessly transmitting, by the wireless power relay, the received power at a second frequency different from the first frequency.

In some examples, the second frequency is less than the first frequency.

In some examples, the second frequency is greater than the first frequency.

In some examples, the received power is not stored in a battery before being transmitted.

In some examples, the received power is stored in, and then retrieved from, a battery before being transmitted.

In some examples, the first power includes first electromagnetic power, the method includes generating a first AC current, having the first frequency, in a receiver antenna of the wireless power relay in response to receiving the first electromagnetic power, and transmitting the received power includes driving a transmitter coil of the wireless power relay with a second AC current, having the second frequency, to generate an alternating magnetic field.

In some examples, the method includes converting the first AC current into the second AC current.

In some examples, the method includes wirelessly transmitting, by a base station separated from the wireless power relay, the power to the wireless power relay.

In some examples, the method includes transmitting, by the wireless power relay, a beacon signal to the base station; and controlling, by the base station, a direction of the power transmitted by the base station based on the beacon signal.

In some examples, the method includes wirelessly receiving, by the electronic device, the power from the wireless power relay, wherein the electronic device includes an implantable medical device.

In some examples, the wireless power relay transmits the received power as an alternating magnetic field.

In some examples, the wireless power relay transmits the received power as electromagnetic power.

In some examples, the wireless power relay transmits the received power as ultrasonic power.

This Summary section introduces some features of nonlimiting and non-exhaustive examples of the present disclosure, and is not intended to limit the scope of the claims.

### BRIEF DESCRIPTION OF THE DRAWINGS

The drawings, together with the specification, illustrate nonlimiting and non-exhaustive examples of the present disclosure.

FIG. 1 is a schematic illustration of a wireless power relay system according to some examples.

FIG. 2 is a schematic illustration of a wireless power relay according to some examples.

FIG. 3 is a flowchart for a method of wirelessly relaying power to an electronic device according to some examples.

### DETAILED DESCRIPTION

Systems and methods for wirelessly relaying power from a power base station to an electronic device will now be described in more detail with reference to the drawings. The systems and methods described herein can improve the efficiency of power transfer, and improve the comfort, convenience, and mobility of a patient, by using a wireless power relay that is configured to receive the power transmitted by the power base station, convert the received power into another form and/or frequency that is more conducive to being received by the electronic device, and transmit the converted power to the electronic device.

It will be understood that, although the terms “first”, “second”, “third”, etc., may be used herein to describe various elements, processes, or other features, these elements, processes, or features should not be limited by these terms. These terms are only used to distinguish one element, process, or feature from another element, process, or feature. Thus, a first element, process, or feature discussed herein could be termed a second element, process, or feature, without departing from the spirit and scope of the present disclosure.

Unless otherwise defined, all terms (including technical and scientific terms) used herein have the same meaning as commonly understood by one of ordinary skill in the art to which the present disclosure belongs. It will be further understood that terms, such as those defined in commonly used dictionaries, should be interpreted as having a meaning that is consistent with their meaning in the context of the relevant art and/or the present specification, and should not be interpreted in an idealized or overly formal sense, unless expressly so defined herein.

The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. It will be further understood that the terms “comprises,” “comprising,” “includes,” and “including,” specify the presence of stated elements, processes, and/or other features, but do not preclude the presence or addition of one or more other elements, processes, and/or features. As used herein, the term “and/or” includes any and all combinations of one or more of the associated listed items. Expressions such as “at least one of,” when preceding a list of elements, modify the entire list of elements and do not modify the individual elements of the list. Further, the use of “may” when describing embodiments of the present disclosure refers to “one or more embodiments of the present disclosure.”

It will be understood that when an element is referred to as being “on”, “connected to”, “coupled to”, “attached to”, or “adjacent to” another element, it can be directly on, connected to, coupled to, attached to, or adjacent to the other element, or one or more intervening element(s) may be present. In contrast, when an element is referred to as being “directly on,” “directly connected to”, “directly coupled to”, “directly attached to”, or “immediately adjacent to” another element, there are no intervening elements present. Similar terms and phrases should be understood in a similar manner to encompass both direct and indirect affiliations between two or more elements being discussed. In addition, it will also be understood that when an element is referred to as being “between” two elements, it can be the only element between the elements, or one or more intervening elements may also be present.

As used herein, the phrase “at least part” includes part or all of the stated item, the phrase “at least partly” includes the stated item partly or entirely, and similar phrases should be interpreted in a similar manner.

As used herein, the term “substantially” and similar terms are used as terms of approximation and not as terms of degree, and are intended to account for the inherent deviations in measured or calculated values that would be recognized by those of ordinary skill in the art.

Also, any numerical range recited herein is intended to include all sub-ranges of the same numerical precision subsumed within the recited range. For example, a range of “1.0 to 10.0” is intended to include all subranges between (and including) the recited minimum value of 1.0 and the recited maximum value of 10.0, that is, having a minimum value equal to or greater than 1.0 and a maximum value equal to or less than 10.0, such as, for example, 2.4 to 7.6. Any maximum numerical limitation recited herein is intended to include all lower numerical limitations subsumed therein and any minimum numerical limitation recited in this specification is intended to include all higher numerical limitations subsumed therein. Accordingly, Applicant reserves the right to amend this specification, including the claims, to expressly recite any sub-range subsumed within the ranges expressly recited herein.

The term “processor” and similar terms are used herein to include any combination of hardware, firmware, and software, employed to process data or digital signals. Processing unit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs).

The wireless power relay, power base station, electronic device, and/or any other relevant devices or components according to embodiments of the present invention described herein may be implemented utilizing any suitable hardware, firmware (e.g. an application-specific integrated circuit), software, or a combination of software, firmware, and hardware. For example, the various components of the wireless power relay, power base station, or electronic device may be formed on one integrated circuit (IC) chip or on separate IC chips. Further, the various components of the wireless power relay, power base station, or electronic device may be implemented on a flexible printed circuit film, a tape carrier package (TCP), a printed circuit board (PCB), or formed on one substrate. Further, the various components of the wireless power relay, power base station, or electronic device may be a process or thread, running on one or more processors, in one or more computing devices, executing computer program instructions and interacting with other system components for performing the various functionalities described herein. The computer program instructions are stored in a memory which may be implemented in a computing device using a standard memory device, such as, for example, a random access memory (RAM). The computer program instructions may also be stored in other non-transitory computer readable media such as, for example, a CD-ROM, flash drive, or the like. Also, a person of skill in the art should recognize that the functionality of various computing devices may be combined or integrated into a single computing device, or the functionality of a particular computing device may be distributed across one or more other computing devices without departing from the scope of the exemplary embodiments of the present invention.

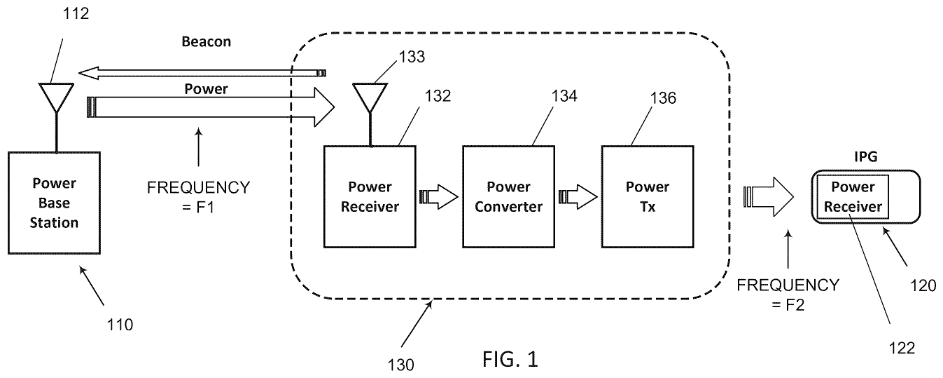

FIG. 1 is a schematic illustration of a wireless power relay system 100 according to some examples. The wireless power relay system 100 may include a power base station 110, an electronic device 120, and a wireless power relay 130 configured to relay power from the power base station 110 to the electronic device 120 in a manner that improves the efficiency of power transferred from the power base station 110 to the electronic device 120.

The electronic device 120 may be, for example, an implantable medical device (IMD), such as an implantable pulse generator (IPG), that is configured to be implanted subcutaneously below the skin of a patient and that relies on an external power source. The power base station 110 may be a stationary or mobile device that is configured to directionally and automatically transmit power to the IPG 120 when the IPG 120 is within the vicinity of (e.g., within the same room) the power base station 110 and without needing to be controlled by the patient. The power base station 110 can therefore conveniently and passively provide power to the IPG 120 while the patient is in the same room as the base station 110. However, the power base station 110 may transmit power having a frequency and form (e.g., high-frequency, far-field electromagnetic power) that experiences high attenuation when passing through biological tissue, which can reduce the efficiency of direct power transfer from the power base station 110 to the IPG 120.

The wireless power relay 130 may be configured to be a wearable device configured to be worn near the IPG 120 implanted within the patient, and the wireless power relay 130 may be configured to convert the power from the power base station 110 into power having a form and/or frequency (e.g., low-frequency, near-field power) that can be more efficiently transmitted through the patient's body to the implanted IPG 120. The wireless power relay 130 can therefore improve the efficiency of power transfer from the power base station 110 to the IPG 120, and improve the comfort, convenience, and mobility of the patient while transferring power to the IPG 120. Additional details and features of the wireless power relay system 100, according to nonlimiting and non-exhaustive examples, are provided below.

The power base station 110 may be configured to transmit first power, having a first frequency F1, to the wireless power relay 130. The first power may be in the form of electromagnetic power. For example, the first power may be RF power, having a frequency in the range of 1 MHz to 300 GHz (e.g., 5.8 GHz), or optical power. In some examples, the power base station 110 includes a first antenna 112 configured to transmit the first power to the wireless power relay 130. The first antenna 112 may be configured to control (e.g., steer) the direction in which the first power is transmitted. For example, the first antenna 112 may include a phased array antenna that includes an array of individually controllable antennas and is configured to use beam forming to steer the direction of the transmitted first power.

As discussed in more detail below, the wireless power relay 130 (or the IPG 120) may be configured to transmit a beacon signal to indicate its location. The power base station 110 may be configured to receive (e.g., via the first antenna 112 or a separate second antenna) and process (e.g., analyze) this beacon signal and transmit the first power based on the beacon signal. For example, the power base station 110 may be configured to control the direction in which the first power is transmitted based on the beacon signal to direct the first power to the wireless power relay 130. This may occur, for example, by using the first antenna 112 to receive the beacon signal and generating the first power using the first antenna as the complex conjugate of the received beacon signal to direct the first power in substantially the opposite direction from which the beacon signal was received by the first antenna 112. When the phased array antenna receives the beacon signal, the individual antennas in the array may receive different portions of the beacon signal that may have different amplitudes and phases. The power base station 110 (e.g., a processor of the power base station 110) may be configured to analyze the different portions of the beacon signal to determine (e.g., calculate) their respective complex conjugates, and then drive the individual antennas with their respective complex conjugate signals to generate the first power that directionally mirrors the beacon signal. This can cause the first power to traverse through the same path(s) that the beacon signal traveled along, thereby directing the first power to the wireless power relay 130. In some examples, the power base station 110 may be configured to generate the first power to have the same frequency as the beacon signal.

The power base station 110 may include a processor (e.g., a control circuit) operatively coupled to the first antenna 112 and configured to receive signals (e.g., the beacon signal) received by the first antenna 112, to process (e.g., analyze) the received signals, and to controllably cause the first antenna 112 to transmit the first power. The power base station 110 may include a power source (e.g., a battery or a wired connector configured to connect to an electrical outlet) that can provide power for generating the first power. In some examples, the power base station 110 includes a memory coupled to the processor and storing instructions that, when executed by the processor, cause the processor to perform one or more processes of the power base station 112 (e.g., any combination of one or more processes of the power base station 110 disclosed herein).

The power base station 110 may be configured as a stationary device that can, for example, be stationarily positioned in a room and transmit the first power to the IPG 120 anywhere in the room (e.g., without requiring a line of sight between the power base station and the patient). A stationary device may refer to a device that requires a wired power connection or that has a size or form factor that does not make it configured to be carried on a patient's person (e.g., carried in a pocket or purse, attachable to clothing articles, etc.). In some other examples, the power base station 110 is configured as a portable device that can be carried with the patient. A portable device may refer to a device that does not require a wired power connection (e.g., a device that includes a battery power source) and that is configured to be carried on the patient's person (e.g., a device that is small enough to be carried in a pocket or purse, attachable to clothing articles, etc.). In some examples, the power base station 110 may have a flat shape and/or be configured to be hung on a wall, for example, behind a painting so that the power base station 110 can be hidden while still providing power to the wireless power relay 130.

As noted above, the electronic device 120 may be an IMD, such as an IPG, that is configured to be implanted subcutaneously under the skin of a patient. In some other examples, the electronic device 120 may be another type of medical or wearable device that is configured to be carried or worn on the exterior of a patient's body or on the patient's clothing. The electronic device 120 will be primarily described herein as an IPG, but the electronic device 120 may be other types of electronic devices and may have features similar to, or the same as, any of the features of the IPG disclosed herein.

The IPG 120 may include a power receiver 122 configured to wirelessly receive second power, having a second frequency F2, from the wireless power relay 130. The IPG 120 may be configured to store the received power (e.g., in a secondary battery of the IPG 120) and/or to use the received power to perform one or more functions of the IPG 120 (e.g., to operate one or more components of the IPG). For example, the IPG 120 may be configured to use the received power to provide electrical stimulation to biological tissue (e.g., to a vagus nerve) of the patient to treat epilepsy, depression, and/or one or more other medical conditions. In some examples, the IPG 120 (e.g., the power receiver 122) is configured to convert the received second power into another form, such as into an AC having the second frequency F2. The IPG 120 may be configured to then convert this AC current into DC current (e.g., via one or more rectifiers) before using the DC current (e.g., to recharge a secondary battery or to provide electrical stimulation to a patient's biological tissue).

In some examples, the power receiver 122 is configured to convert a near field alternating magnetic field into the AC current. For example, the power receiver 122 may include a receiver coil configured to generate the AC current in response to receiving the alternating magnetic field. In some examples, the power receiver 122 is configured to convert far field electromagnetic power (e.g., RF power, optical power, etc.) into the AC current. For example, the power receiver 122 may include a receiver antenna configured to generate the AC current in response to receiving the far field electromagnetic power. In some examples, the power receiver 122 is configured to convert ultrasonic power into the AC current. For example, the power receiver 122 may include an acoustic transducer (e.g., piezoelectric transducer) configured to generate the AC current in response to receiving ultrasound vibrations.

The IPG 120 may include, or omit, a battery, which may depend on the application and purpose of the IPG 120. For example, if the IPG 120 is only contemplated to be used at times when the patient is likely to be near the power base station 110 (e.g., as in the case when the IPG 120 is only used at night when the patient is sleeping at home), then the IPG 120 can omit the rechargeable battery and rely on power received by the power base station 110. However, if it is important for the IPG 120 to always be operatable (e.g., as in the case of treating pain or epilepsy), including when the patient is away from the power base station 110, then it is advantageous for the IPG 120 to include a rechargeable battery so that the IPG can be used even when the patient is not in the vicinity of the power base station 110. Because a battery can occupy a substantial amount of space in an IPG, omitting the battery can reduce the size of the IMD, which can make implantation of the IMD easier and less invasive. Omitting the battery can also be beneficial because the patient will not need to spend a potentially long time (e.g., an hour or more) recharging the battery. In some examples, the IPG 120 may exclude the rechargeable battery, but the power base station 110 may be configured as a portable device so that the patient can carry the power base station 110 with him or her.

The IPG 120 may include other components, such as a processor (e.g., a control circuit), a memory, and/or a communication transmitter (e.g., communication transceiver). The communication transmitter (e.g., a Bluetooth radio) may be configured to transmit information to external devices, such as the wireless power relay 130 and/or to the power base station 110. The processor may be operatively coupled to other components of the IPG 120 and configured to control operations of the IPG 120. The memory may store instructions that, when executed by the processor of the IPG 120, cause the processor to perform one or more processes of the IPG 120 (e.g., any combination of one or more processes of the IPG 120 disclosed herein).

The wireless power relay 130 may be configured to wirelessly receive the first power, having the first frequency F1, from the power base station 110 and to use the first power to generate and transmit the second power, having the second frequency F2, to the IPG 120. For example, the wireless power relay 130 may be configured to convert the first power into the second power, and this may entail changing the frequency and/or form of the received power before transmission. Thus, the wireless power relay 130 may be configured as a power relay and a power converter.

The first and second frequencies F1 and F2 may be the same or different from one another. For example, the second frequency F2 may be higher or lower than the first frequency F1. In some examples, the second frequency F2 may be in the range of 1 kHz to 1 MHz (e.g., 126.31 kHz). In some other examples, the second frequency F2 may be in a higher range, such as between 1 MHz to 300 THz or in the range of optical frequencies. Lower frequency magnetic and electromagnetic power can travel through a patient's body with less attenuation than higher frequency magnetic and electromagnetic power. Thus, it can be advantageous for the wireless power relay 130 to transmit the second power with a relatively low frequency to improve the efficiency of power transfer from the wireless power relay 130 to the IPG 120. At the same time, it can be advantageous for the power base station 110 to transmit the first power with a relatively high frequency to increase power transfer from the power base station 110 to the wireless power relay 130. The combination of a relatively high first frequency F1 and a relatively low second frequency F2 can therefore improve the overall efficiency of the wireless power relay system 100.

In more detail, the wireless power relay 130 may include a power receiver 132 configured to wirelessly receive the first power from the power base station 110, a power transmitter 136 configured to wirelessly transmit the second power to the IPG 120, and a power converter 134 coupled between (e.g., electrically coupled between) the power receiver 132 and the power transmitter 136 and configured to convert the frequency and/or form of power.

In some examples, the power receiver 132 includes an antenna 133 configured to generate a first AC current, having the first frequency F1, in response to wirelessly receiving the first power. The antenna 133 may be configured to also transmit (e.g., occasionally or periodically transmit) the beacon signal to the power base station 110 to indicate the location of the wireless power relay 130. In some other examples, a separate antenna is used to transmit the beacon signal. The antenna 133 may be a phased array antenna in some examples.

The power transmitter 136 may be configured to transmit the second power in any suitable form, and the form of the second power may be the same as, or different from, the form of the first power received by the power receiver 132. In some examples, the power transmitter 136 is configured to transmit an alternating magnetic field that alternates at the second frequency F2. For example, the power transmitter 136 may include one or more transmitter coils configured to generate the alternating magnetic field in response to being driven with a second AC current having the second frequency F2. In some examples, the power transmitter may include two or more transmitter coils, which may be arranged in a quadrature or orthogonal configuration. In some examples, the power transmitter 136 is configured to transmit electromagnetic power (e.g., optical power, RF power, or lower frequency power). For example, the power transmitter 136 may include a transmitter antenna configured to convert the second AC current into the electromagnetic power having the second frequency F2. In some examples, the power transmitter 136 is configured to generate ultrasonic power vibrating at the second frequency F2. For example, the power transmitter 136 may include an acoustic transducer, such as a piezoelectric transducer configured to convert the second AC current into ultrasonic vibrations vibrating at the second frequency F2.

The power converter 134 may be configured to convert the frequency and/or form of power input into the power converter 134 from the power receiver 132, and to provide the converted power to the power transmitter 136. For example, the power converter 134 may be configured to convert the first AC current, received from the power receiver 132 and having the first frequency F1, into the second AC current, having the second frequency F2, and transmit the second AC current to the power transmitter 136. This may entail first converting the first AC current into DC current, and then converting the DC current into the second AC current. Other types of power converters and/or frequency converters, and other processes for converting the form and/or frequency of power, may also be used in the power converter 134. In some examples, the power converter may form an integral component with one or both of the power receiver 132 and the power transmitter 136.

The power receiver 132 and the power transmitter 136 may be respectively at least partly positioned in separate first and second compartments, and a power separator component may be positioned at least partly between the first and second compartments (e.g., at least partly between the power receiver 132 and the power transmitter 136). The power separator component may be configured to at least partly block the first power received from propagating into the second compartment and/or to at least partly block the second power from propagating into the first compartment. This can improve the independent functionality of the power receiver 132 and the power transmitter 136 by reducing interference (e.g., cross-talk) between the two. In some examples, the power separator component includes an electrically conductive material (e.g., an electrically conductive wall) that at least partly surrounds the first compartment and/or at least partly surrounds the second compartment (e.g., that extends between the power receiver 132 and the power transmitter 136). This can at least partly block magnetic fields and electromagnetic fields from crossing between the first and second compartments. In some examples, the power separator component includes an acoustic separator (e.g., a material have particular physical properties) configured to reduce (e.g., to dampen) ultrasonic vibrations and at least partly surrounding the first compartment and/or at least partly surrounding the second compartment (e.g., extending between the power receiver 132 and the power transmitter 136). This can reduce ultrasonic energy from crossing between the first and second compartments.

In some examples, the wireless power relay 130 is configured to wirelessly receive, convert, and wirelessly transmit power without storing power in an energy storage device, such as a battery (e.g., a rechargeable battery) or supercapacitor. For example, the wireless power relay 130 does not include a rechargeable battery (or any battery) or supercapacitor in some examples. As used herein, an energy storage device, such as a battery may refer to a component configured to controllably release electrical power (e.g., under the control of a processor or control circuit) and that includes one or more electrochemical cells configured to undergo redox reactions to generate the electrical power. A battery thus does not include components such as a passive capacitor in a circuit (e.g., a capacitor in an LC circuit). Thus, the wireless power relay 130 may be configured to passively relay the power received from the power base station 110 to the IPG 120 without storing the power in a battery. This can reduce the number of components, and complexity of operations of, the wireless power relay 130 and can also significantly reduce the size and weight of the wireless power relay 130, which can make the wireless power relay 130 easier and more comfortable for the patient to wear or carry.

However, in some other examples, the wireless power relay 130 may include a battery (e.g., a primary or secondary battery). The wireless power relay 130 may be configured to store at least some of the power received from the power base station 110 in the secondary battery. This has the benefit of allowing the wireless power relay 130 to continue to transmit the second power, using power stored in the battery, for at least some time after the patient leaves the vicinity of the power base station 110 or in instances where the power base station 110 ceases to transmit power (e.g., due to malfunction, power failure, etc.).

The wireless power relay 130 may have a relatively small size and low weight. For example, the wireless power relay 130 may have a compact puck shape. In some examples, a largest dimension of the wireless power relay 130 may be less than 6 inches, less than 5 inches, less than 4 inches, less than 3 inches, less than 2 inches, or less than 1 inch. The small size and weight of the wireless power relay 130 may be achievable due to the reduced number of components included in the wireless power relay 130. For example, the absence of a battery may significantly reduce the size and weight of the wireless power relay 130.

In some examples, the wireless power relay 130 is configured to attach to the skin or of a patient or to a clothing article (e.g., an upper garment). For example, the wireless power relay 130 may include an adhesive at an outer surface that is configured to attach the wireless power relay 130 to the skin of the patient or to a garment of the patient. The adhesive may be a biocompatible adhesive to accommodate attachment of the wireless power relay 130 to the patient's skin. In some examples, the wireless power relay 130 is configured to attach to the patient's clothing, for example, via a clip, pin, or hook-and-loop fastener. The wireless power relay system 100 may include a wearable garment configured to support and position the wireless power relay 130 in proximity to the implanted IPG 120. For example, the wearable garment may include an attachment device (e.g., a snap device, hook-and-loop fastener, etc.), for attaching to the wireless power relay 130, or a pocket shaped and sized to receive the wireless power relay 130. The attachment device may be positioned or positionable at a location likely to be adjacent to the IPG 120 implanted within the patient.

The wireless power relay 130 may include other components, such as a processor (e.g., control circuit), a memory, a communication receiver (e.g., a communication transceiver), and/or a user interface. The communication receiver (e.g., a Bluetooth radio) may be configured to receive information from external devices, such as from the IPG 120. The user interface may include a display (e.g., a touch display), a speaker, and/or a microphone, and may be configured to allow a user (e.g., the patient, a physician, etc.) to interact with (e.g., control, set parameters of, etc.) the wireless power relay 130 and/or the IPG 120. The processor may be operatively coupled to other components of the wireless power relay 130 and configured to control operations of the wireless power relay 130. The memory may store instructions that, when executed by the processor of the wireless power relay 130, cause the processor to perform one or more processes of the wireless power relay 130 (e.g., any combination of one or more processes of the wireless power relay 130 disclosed herein). In some examples, a portion of the first power may be siphoned off and used to power components or operations of the wireless power relay 130 (e.g., to power the processor of the wireless power relay 130).

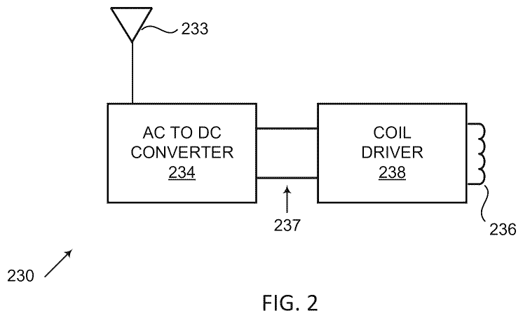

FIG. 2 is a schematic illustration of a wireless power relay 230 according to some examples. The wireless power relay 130 of FIG. 1 may include the wireless power relay 230 of FIG. 2. In some examples, the wireless power relay 230 includes a receiver antenna 233, a transmitter coil 236, an AC to DC converter 234 coupled between the receiver antenna 233 and the transmitter coil 236, a coil driver 238 electrically coupled between the AC to DC converter 234 and the transmitter coil 236, and electrical connection(s) 237 electrically coupled between the AC to DC converter 234 and the coil driver 238.

The receiver antenna 233 may be configured to generate a first AC current, having a first frequency, in response to receiving alternating electromagnetic power (e.g., RF power or optical power) having the first frequency. The AC to DC converter 234 (e.g., an electrical current rectifier) may be configured to receive the first AC current from the receiver antenna 233 and convert the first AC current to DC current. The coil driver 238 may be configured to receive, via the electrical connection(s) 237, the DC current from the AC to DC converter 234 and use the DC current to drive the transmitter coil 236 to generate an alternating magnetic field alternating at a second frequency different from (e.g., higher than or lower than) the first frequency. For example, the coil driver 238 may include a DC to AC converter (e.g., a power inverter) that is configured to convert the DC current into second AC current, having the second frequency, and to drive the transmitter coil 236 with the second AC current. The transmitter coil 236 may be configured, in response to being driven with the second AC current, to generate the alternating magnetic field. The alternating magnetic field may be inductively received by the IPG and used to power or charge the IPG.

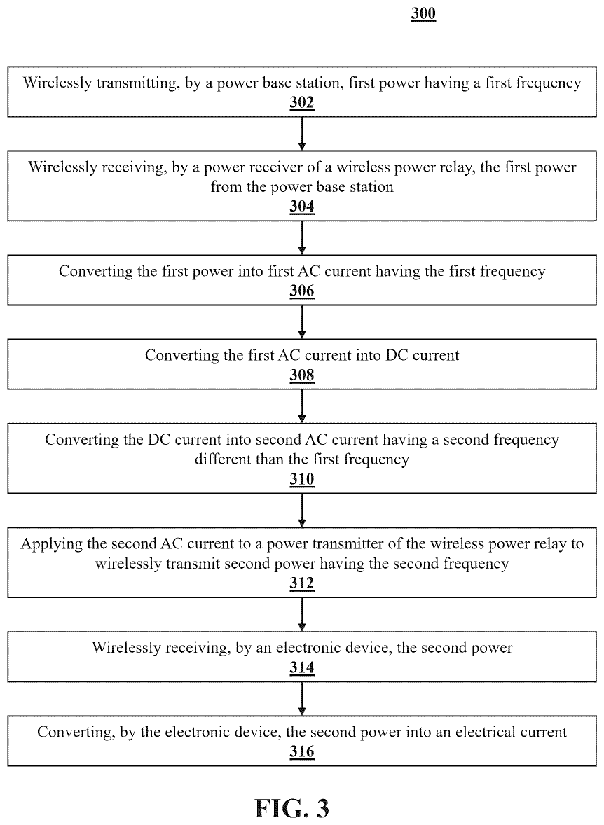

FIG. 3 is a flowchart for a method 300 of wirelessly relaying power to an electronic device according to some examples. The method 300 may be performed by any wireless power relay system within the scope of the present disclosure.

The method 300 may include a first process 302 of wirelessly transmitting, by a power base station, first power. The first power may include, for example, far field electromagnetic power and may be directionally controlled by the power base station to be transmitted to a wireless power relay. The power base station may control the direction of the first power based on a beacon signal transmitted by the wireless power relay and received by the power base station. The method 300 may include a second process 304 of wirelessly receiving, by a power receiver of the wireless power relay, the first power from power base station. The method 300 may include a third process 306 of converting the first power into first AC current having the first frequency, a fourth process 308 of converting the first AC current into DC current, and a fifth process 310 of converting the DC current into second AC current having a second frequency different from (e.g., lower than or higher than) the first frequency. A sixth process 312 may include applying the second AC current to a power transmitter of the wireless power relay to wirelessly transmit second power, having the second frequency, to an electronic device (e.g., an IMD). The second power may include an alternating magnetic field, electromagnetic power, or ultrasonic power. During a seventh process 314, the electronic device may wirelessly receive the second power from the wireless power relay. During an eighth process 316, the electronic device may convert the second power into an electrical current for use in the electronic device.

Although some methods for wirelessly relaying power to an electronic device have been discussed with reference to FIG. 3, the present disclosure is not limited thereto. Systems for wirelessly relaying power to an electronic device, and processes performed by such systems, have been described herein with reference to FIGS. 1 and 2, and the present disclosure includes all methods for wirelessly relaying power to an electronic device that include any combination of such processes in any suitable order.

Features from an embodiment, or from multiple embodiments, described in the present disclosure may be combined with each other, partially or entirely, and may be technically interlocked and operated in various ways, and the embodiments described herein may be implemented independently of each other or in conjunction with each other.

Although specific embodiments are described herein, the scope of the technology is not limited to those specific embodiments. Moreover, while different embodiments may be described separately, such embodiments and examples may be combined with one another in implementing the technology described herein. One skilled in the art will recognize other embodiments or improvements that are within the scope and spirit of the present technology. Therefore, the specific elements, features, and processes are disclosed only as example embodiments. The scope of the technology is defined by the following claims and any equivalents thereof.

What is claimed is:

1. A wireless power relay for wirelessly relaying power to an electronic device, the wireless power relay comprising:
a power receiver configured to wirelessly receive power;
a power transmitter configured to wirelessly transmit power; and
a power converter operatively coupled between the power receiver and the power transmitter, the power converter being configured:
to convert first power at a first frequency wirelessly received by the power receiver, and
to apply the converted power to the power transmitter such that the power transmitter wirelessly transmits second power at a second frequency different from the first frequency.

2. The wireless power relay of claim 1, wherein the second frequency is lower than the first frequency.

3. The wireless power relay of claim 1, wherein the second frequency is higher than the first frequency.

4. The wireless power relay of claim 1, wherein the electronic device comprises an implantable medical device.

5. The wireless power relay of claim 4, wherein the wireless power relay comprises a biocompatible adhesive configured to attach the wireless power relay to the skin of a patient.

6. A wireless power relay system, comprising:
the wireless power relay of claim 4, and
a wearable garment configured to support and position the wireless power relay in proximity to the implantable medical device.

7. The wireless power relay of claim 1, wherein the power converter is configured to convert the first power and to apply the converted power to the power transmitter without storing power in a battery.

8. The wireless power relay of claim 1, wherein the wireless power relay does not include a battery.

9. The wireless power relay of claim 1, wherein the wireless power relay includes a battery.

10. The wireless power relay of claim 1, wherein the power receiver comprises a receiver antenna, and the power transmitter comprises a transmitter coil.

11. The wireless power relay of claim 10, wherein the power converter is configured:
to convert a first AC current, having the first frequency and being generated in the receiver antenna in response to the receiver antenna wirelessly receiving the first power, to a second AC current having the second frequency; and
to provide the second AC current to the transmitter coil to cause the transmitter coil to wirelessly transmit the second power.

12. The wireless power relay of claim 1, wherein the power receiver and the power transmitter are respectively positioned in first and second compartments that are at least partially separated by a power separator configured to at least partially separate the first power from the second power.

13. The wireless power relay of claim 12, wherein at least one of the first power or the second power comprises electromagnetic power or magnetic power, and the power separator comprises an electrically conductive element configured to at least partially block electromagnetic power and magnetic power from transferring between the first and second compartments.

14. The wireless power relay of claim 12, wherein the second power comprises ultrasonic power, and the power separator comprises an acoustic separator configured to at least partially block ultrasonic power from transferring from the second compartment into the first compartment.

15. The wireless power relay of claim 1, further comprising a beacon transmitter configured to wirelessly transmit a beacon signal.

16. A wireless power transfer system, comprising:
the wireless power relay of claim 1; and
a base station configured to wirelessly transmit the first power at the first frequency to the wireless power relay.

17. The wireless power transfer system of claim 16, further comprising the electronic device, the electronic device being configured to wirelessly receive the second power from the wireless power relay.

18. The wireless power transfer system of claim 16, wherein the base station is configured to control a direction of the first power wirelessly transmitted by the base station.

19. The wireless power transfer system of claim 18, wherein the wireless power relay comprises a beacon transmitter configured to wirelessly transmit a beacon signal, and
wherein the base station comprises a phased array antenna configured to wirelessly receive the beacon signal, the base station being configured to wirelessly transmit the first power, via the phased array antenna, based on the beacon signal.

20. A wireless power transfer system, comprising:
the wireless power relay of claim 1; and
the electronic device, the electronic device comprising an implantable medical device configured to wirelessly receive the second power from the wireless power relay.

21. The wireless power transfer system of claim 20, wherein the implantable medical device is an implantable pulse generator configured to provide stimulation to tissue.

22. The wireless power relay of claim 1, wherein the power receiver comprises an antenna configured to receive electromagnetic power.

23. The wireless power relay of claim 1, wherein the power transmitter comprises a transmitter coil configured to generate an alternating magnetic field.

24. The wireless power relay of claim 1, wherein the power transmitter comprises a transmitter antenna configured to generate electromagnetic power.

25. The wireless power relay of claim 1, wherein the power transmitter comprises an ultrasonic transducer configured to generate ultrasonic power.

26. A wireless power relay for wirelessly relaying power to an electronic device, the wireless power relay comprising:
a receiver antenna configured to generate a first AC current in response to receiving first electromagnetic power;
a transmitter coil configured to generate an alternating magnetic field in response to being driven with a second AC current; and
a frequency converter operatively coupled between the receiver antenna and the transmitter coil, the frequency converter being configured:
to convert the first AC current, having a first frequency, to the second AC current having a second frequency different from the first frequency, and
to drive the transmitter coil with the second AC current.

27. The wireless power relay of claim 26, wherein the electronic device comprises an implantable medical device.

28. The wireless power relay of claim 26, wherein the second frequency is lower than the first frequency.

29. The wireless power relay of claim 26, wherein the second frequency is higher than the first frequency.

30. The wireless power relay of claim 26, wherein the wireless power relay does not comprise a battery.

31. The wireless power relay of claim 26, wherein the wireless power relay comprises a battery.

32. The wireless power relay of claim 26, wherein the wireless power relay comprises a biocompatible adhesive configured to attach the wireless power relay to the skin of a patient.

33. A wireless power transfer system, comprising:
the wireless power relay of claim 26; and
a base station configured to transmit the first electromagnetic power to the wireless power relay.

34. The wireless power transfer system of claim 33, further comprising an implantable medical device configured to wirelessly receive the alternating magnetic field and to convert the alternating magnetic field into an electric current.

35. A wireless power transfer system, comprising:
the wireless power relay of claim 26; and
an implantable pulse generator configured to wirelessly receive the alternating magnetic field.

36. A method for wirelessly relaying power to an electronic device, the method comprising:
wirelessly receiving, by a wireless power relay, power having a first frequency; and
wirelessly transmitting, by the wireless power relay, the received power at a second frequency different from the first frequency.

37. The method of claim 36, wherein the second frequency is less than the first frequency.

38. The method of claim 36, wherein the second frequency is greater than the first frequency.

39. The method of claim 36, wherein the received power is not stored in a battery before being transmitted.

40. The method of claim 36, wherein the received power is stored in, and then retrieved from, a battery before being transmitted.

41. The method of claim 36, wherein the first power comprises first electromagnetic power,
wherein the method comprises generating a first AC current, having the first frequency, in a receiver antenna of the wireless power relay in response to receiving the first electromagnetic power, and
wherein transmitting the received power comprises driving a transmitter coil of the wireless power relay with a second AC current, having the second frequency, to generate an alternating magnetic field.

42. The method of claim 41, comprising converting the first AC current into the second AC current.

43. The method of claim 36, comprising wirelessly transmitting, by a base station separated from the wireless power relay, the power to the wireless power relay.

44. The method of claim 43, comprising:
transmitting, by the wireless power relay, a beacon signal to the base station; and
controlling, by the base station, a direction of the power transmitted by the base station based on the beacon signal.

45. The method of claim 36, comprising wirelessly receiving, by the electronic device, the power from the wireless power relay, wherein the electronic device comprises an implantable medical device.

46. The method of claim 36, wherein the wireless power relay transmits the received power as an alternating magnetic field.

47. The method of claim 36, wherein the wireless power relay transmits the received power as electromagnetic power.

48. The method of claim 36, wherein the wireless power relay transmits the received power as ultrasonic power.
